SEQUENCE LISTING (13 sequences)

SEQ ID NO: 1 - DNA, 29 bp, Artificial Sequence
  Description of Artificial Sequence PCR primer for E. coli argU gene
gacactagtaatcagacgcggtcgttcac

SEQ ID NO: 2 - DNA, 32 bp, Artificial Sequence
  Description of Artificial Sequence PCR primer for E. coli argU gene
gacgacgacaagaatcagacgcggtcgttcac

SEQ ID NO: 3 - DNA, 29 bp, Artificial Sequence
  Description of Artificial Sequence PCR primer for E. coli argU gene
ctgccatggtggaggatataaagaaggcg

SEQ ID NO: 4 - DNA, 29 bp, Artificial Sequence
  Description of Artificial Sequence PCR primer for E. coli ileY gene
cagccatggccttgaaatggcgttagtca

SEQ ID NO: 5 - DNA, 29 bp, Artificial Sequence
  Description of Artificial Sequence PCR primer for E. coli ileY gene
gacactagtccttgaaatggcgttagtca

SEQ ID NO: 6 - DNA, 29 bp, Artificial Sequence
  Description of Artificial Sequence PCR primer for E. coli ileY gene
cagtctagatcatcatgtttattgcgtgg

SEQ ID NO: 7 - DNA, 29 bp, Artificial Sequence
  Description of Artificial Sequence PCR primer for E. coli ileY gene
gacctcgagtcatcatgtttattgcgtgg

SEQ ID NO: 8 - DNA, 29 bp, Artificial Sequence
  Description of Artificial Sequence PCR primer for E. coli leuW gene
cagtctagagaatcccgtcgtagccacca

SEQ ID NO: 9 - DNA, 30 bp, Artificial Sequence
  Description of Artificial Sequence PCR primer for E. coli leuW gene
gacctcgagggcatccgatcaacgctttct

SEQ ID NO: 10 - DNA, 29 bp, Artificial Sequence
  Description of Artificial Sequence PCR primer for E. coli proL gene
gacgtcgacgtgctgacagacgagaagcg

SEQ ID NO: 11 - DNA, 29 bp, Artificial Sequence
  Description of Artificial Sequence PCR primer for E. coli proL gene
gacctcgagggtgtggtctggacgttctg

SEQ ID NO: 12 - DNA, 29 bp, Artificial Sequence
  Description of Artificial Sequence PCR primer for E. coli glyU gene
ctgccatggggcacttgctaaggagagcg

SEQ ID NO: 13 - DNA, 33 bp, Artificial Sequence
  Description of Artificial Sequence PCR primer for E. coli glyU gene
ggaacaagagggcgtgttttcctgggttgttac

(19) United States
(12) Patent Application Publication
(10) Pub. No.: US 2004/0214281 A1
(43) Pub. Date: Oct. 28, 2004

(54) Methods and compositions for high level expression of a heterologous protein with poor codon usage

(76) Inventor: Carsten-Peter Carstens, La Jolla, CA (US)

Correspondence Address: PALMER & DODGE, LLP, KATHLEEN M. WILLIAMS / STR, 111 HUNTINGTON AVENUE, BOSTON, MA, 02199, US

(21) Appl. No.: 10/777,010

(22) Filed: Feb. 11, 2004

Related U.S. Application Data
(63) Continuation of application No. 09/492,590, filed on Jan. 27, 2000.
(60) Provisional application No. 60/117,355, filed on Jan. 27, 1999.

(51) Int. Cl. C12P 21/02; C12N 1/21; C12N 15/74

(52) U.S. Cl. 435/69.1; 435/488; 435/252.33

(57) ABSTRACT

High level expression of heterologous proteins in host cells is frequently limited by the presence of rarely utilized codons, due to depletion of the internal tRNA pool and stalling of translation. This invention provides expression host cells generated by introduction of a vector for the expression of an array of tRNA genes that are rare in the host cells. The modified host cells are capable of efficiently supporting expression of selected recombinant genes which otherwise would be limited by the presence of rare codons.

### FIELD OF THE INVENTION

The invention is related to the area of expression of heterologous proteins in bacteria. In particular it is related to the field of codon usage.

### BACKGROUND OF THE INVENTION

Expression of heterologous proteins in bacteria such as E. coli has become a standard procedure in most molecular biology laboratories and a cornerstone of production in the biotechnology industry. The most frequent problems encountered in bacterial expression systems are the insolubility of the induced protein and the poor efficiency of expression. Expression of heterologous proteins in bacteria can be seriously compromised due to the different codon preference displayed in other organisms. The codon frequencies in E. coli and other organisms are compared in Table 1.

Forced high level expression of a gene that uses a codon rarely utilized in the host bacterial cell can lead to depletion of the cognate tRNA species and subsequent stalling of the translation process (Kane, J. F., “Effects of rare codon clusters on high-level expression of heterologous proteins in Escherichia coli,” Curr. Opin. Biotechnol. 6:494-500 (1995); Bonekanp, F., Andersen, H. D., Christensen, T., Jensen, K. F, “Codon-defined ribosomal pausing in Escherichia coli detected by using the pyre attenuator to probe the coupling between transcription and translation,” Nucleic Acids Res. 13:4113-23 (1985)). Stalling of the translation process may result in premature degradation of mRNA (Deana, A., Ehrlich, R., Reiss, C., “Silent mutations in the Escherichia coli ompa leader peptide region strongly affect transcription and translation in vivo,” Nucleic Acids Res. 26:4778-4782 (1998)).

Analysis of positional effects of rare codons on protein expression in E. coli has demonstrated that these codons are a particular problem if the same codon appears frequently, consecutively, and close to the N-terminus (Rosenberg, A. H., Goldman, E., Dunn J. J., Studier, F. W., Zubay, G., “Effects of consecutive AGG codons on translation in Escherichia coli, demonstrated with a versatile codon test system,” J. Bacteriol. 175:716-22 (1993); Goldman, E., Rosenberg, A. H., Zubay, G., Studier, F. W., “Consecutive low-usage leucine codons block translation only when near the 5′ end of a message in Escherichia coli,” J. Mol. Biol. 245:467-73 (1995); Degryse, E., “Influence of the second and third codon on the expression of recombinant hirudin in E. coli,” FEBS Lett. 269: 244-6 (1990)).

The effects of rare E. coli codons on high level expression are particularly well documented in the case of the arginine codons AGA and AGG, which are the rarest codons in E. coli (reviewed in Kane (1995) supra). However, other codons such as CGA (arg), CUA (leu), AUA (ile), CCC (pro), and the glycine codons GGA and GGG also have been shown to affect protein expression (Kane (1995) supra; Rosenberg et al., (1993) supra; Goldman et al., (1995) supra; Del Tito, B. J. Jr., Ward, J. M., Hodgson, J., Gershater, C. J., Edwards, H., Wysocki, L. A., Watson, F. A, Sathe, G., Kane, J. F., “Effects of a minor isoleucyl tRNA on heterologous protein translation in Escherichia coli,” J. Bacteriol. 177:7086-91 (1995)).

In addition to effects on yield, the quality of the protein can be affected by the presence of rare codons. Stalling during translation can lead to frameshifts or skipping of a particular codon (Spanjaard, R. A., Chen, K., Walker, J. R., van Duin, J., “Frameshift suppression at tandem AGA and AGG codons by cloned tRNA genes: assigning a codon to argu tRNA and T4 tRNA(Arg),” Nucleic Acids Res. 18:5031-6 (1990); Kane J. F., Violand, B. N., Curran, D. F., Staten, N. R., Duffin, K. L., Bogosian, G., “Novel in-frame two codon translational hop during synthesis of bovine placental lactogen in a recombinant strain of Escherichia coli,” Nucleic Acids Res. 20:6707-12 (1992)).

Another potentially significant problem is the misincorporation of lysine for arginine at AGA or AGG codons (Calderone, T. L., Stevens, R. D., Oas, T. G., “High-level misincorporation of lysine for arginine at AGA codons in a fusion protein expressed in Escherichia coli,” J. Mol. Biol. 262:407-12 (1996)). Misincorporation rates can reach 43% at a given residue (Forman, M. D., Stack, R. F., Masters, P. S., Hauer, C. R., Baxter, S. M., “High level, context dependent misincorporation of lysine for arginine in Saccharomyces cerevisiae al homeodomain expressed in Escherichia coli,” Protein Sci. 7:500-3 (1998)), which may cause problems if the protein is used for structural analysis.

Expression of E. coli genes for rare codons in bacterial host cells has been found to increase the expression of heterologous proteins requiring the rare codons. Several laboratories have shown that transfection of host cells with the argU gene, which encodes a tRNA species utilizing the rare AGA/AGG codons, together with a gene for a recombinant protein requiring these codons can increase heterologous protein expression. Brinkmann et al., Gene 85:109-14 (1989); Hua et al., Biochem. Mol. Biol. Int. 32:537-43 (1994); Chen et al., Genes Dev. 8:2641-52 (1994); and Garcia et al., Ann. N.Y. Acad. Sci. 782:79-86 (1996). A similar effect was observed for transfection with the ileX gene, which utilizes the rare isoleucine codon AUA. Del Tito, Jr., et al., J. Bacteriol. 177:7086-7091 (1995). Coexpression with both argU and ileX genes has also been shown to increase the expression of certain bacterial and viral recombinant proteins. Del Tito, Jr., et al., supra; Kim et al., Biotech. Lett. 20:207-210 (1998). Overexpression of tRNA genes can be deleterious to the host cell, however. Rojiani et al., Proc. Nat. Acad. Sci. U.S.A. 87:1511-1515 (1990); Sharp et al., Nucleic Acids Res. 14:7737-7749 (1986).

There remains a need in the art for methods and reagents which permit high level expression of heterologous recombinant proteins, particularly eukaryotic proteins, whose expression is limited by codon usage in the host cell. Furthermore, there is a need for host cells and methods of making them which can overcome poor usage of several rare codons simultaneously.

### SUMMARY OF THE INVENTION

It is an object of the invention to provide methods and reagents which make possible the high level expression of heterologous recombinant proteins whose codon usage differs from that of the host cells. This and other objects of the invention are provided by one or more of the embodiments described below.

One embodiment of the invention provides a host cell comprising a recombinant DNA molecule which comprises an array of three or more tRNA genes. The tRNA genes correspond to codons that are rarely used in the host cell. In some versions of this embodiment, the tRNA genes correspond to codons present in a gene from an organism other than the host cell. One or more of the tRNA genes can be heterologous to the host cell. The host cell can further comprise a recombinant DNA molecule which comprises a gene encoding a protein of interest.

Preferably, the array of tRNA genes is operatively associated with a transcription control element operative in the host cell. For example, the transcription control element can be activated by isopropylthio-β-galactoside (IPTG), which activates transcription of the tRNA genes. Transcription of the tRNA genes can be controlled by a promoter for T7 RNA polymerase.

The host cell may be protease deficient; for example, the host cell can be deficient in Lon and OmpT proteases. In some host cells of this embodiment, the recombinant DNA molecule comprises a medium to low copy number vector. The medium to low copy number vector can comprise a particular array of tRNA genes. For example, the array can comprise the E. coli genes argU and ileY. The array can also comprise the E. coli genes argU, ileY, and leu W. The array can also comprise the E. coli genes argU, ileY, leu W, and proL. The array can comprise the E. coli genes argU and proL.

Some host cells of this embodiment are bacterial cells, for example E. coli cells. Some E. coli cells of this embodiment have the Hte (high transformation efficiency) phenotype. Some E. coli cells of this embodiment are EndA1 deficient, and some are RecA positive.

Another embodiment of the invention provides a vector that replicates in a host cell. The vector comprises an array of three or more tRNA genes which correspond to codons that are rarely used in said host cell. Some vectors of this embodiment comprise a restriction endonuclease site located between the coding sequences of any two tRNA genes.

Still another embodiment of the invention provides a method of producing a protein of interest. The method comprises the step of culturing a host cell that comprises a recombinant DNA molecule. The recombinant DNA molecule comprises an array of three or more tRNA genes which correspond to codons that are rarely used in the host cell. The codons are present in the gene for the protein of interest. The conditions of culturing the host cell are sufficient to produce the protein of interest.

Yet another embodiment of the invention provides a kit comprising a vector, a host cell, and packaging materials therefor. The vector replicates in the host cell and comprises an array of three or more tRNA genes corresponding to codons which are rarely used in the host cell. In some kits of this embodiment, the vector comprises a restriction endonuclease site located between the coding sequences of any two tRNA genes.

Another embodiment of the invention provides a host cell comprising a recombinant DNA molecule which comprises a set of two tRNA genes. The set does not consist of the pair of E. coli genes argu and ileX. The tRNA genes correspond to codons that are rarely used in the host cell.

In some host cells of this embodiment, the set consists of tRNA genes that specify codons for arginine and proline; for example, the set can comprise the E. coli tRNA genes argU and proL.

In some host cells of this embodiment, the set consists of tRNA genes that specify codons for arginine and leucine or arginine and glycine.

In some host cells of this embodiment, the set consists of tRNA genes that specify codons for proline and leucine, proline and isoleucine, or proline and glycine.

In some host cells of this embodiment, the set comprises tRNA genes that specify codons for leucine and isoleucine or leucine and glycine.

In some host cells of this embodiment, the set comprises tRNA genes that specify codons for isoleucine and glycine.

A further embodiment of the invention provides a vector that replicates in a host cell. The vector comprises an array of tRNA genes which correspond to codons that are rarely used in said host cell. The array does not consist of only the pair of E. coli genes argU and ileX.

Yet another embodiment of the invention provides a method of producing a protein of interest. The method comprises the step of culturing a host cell that comprises a recombinant DNA molecule. The recombinant DNA molecule comprises an array of tRNA genes which correspond to codons that are rarely used in the host cell, where the array does not consist of only the pair of E. coli genes argU and ileX. The codons are present in the gene for the protein of interest. The conditions of culturing the host cell are sufficient to produce the protein of interest.

Another embodiment of the invention provides a kit comprising a vector, a host cell, and packaging materials therefor. The vector replicates in the host cell and comprises an array of tRNA genes corresponding to codons which are rarely used in the host cell, and where the array does not consist of the pair of E. coli genes argU and ileX.

Further features and advantages of the invention will become more fully apparent in the following description of the embodiments and drawings thereof, and from the claims.

### BRIEF DESCRIPTION OF THE DRAWINGS

FIG. 1 displays pACYC-LIC expression vectors for E. coli tRNA gene arrays. All tRNA gene arrays were cloned between the SpeI and XhoI sites of pACYC-LIC except for the RG array, which was cloned by ligation independent cloning between the LIC sites of pACYC-LIC, indicated by boxes flanking the SpeI and XhoI sites. The tRNA genes were amplified by PCR from E. coli K12 genomic DNA. Arrays were generated by ligation of the individual tRNA fragments and re-amplification using flanking primers for the specific array. The restriction sites demarcate the junctions between tRNA gene fragments. Expression is driven by the tetracycline resistance gene promoter; however, each fragment may contain its own promoter. pACYC-based plasmids are medium to low copy number plasmids compatible with colE1 based plasmids.

FIG. 2 demonstrates rescue of the expression of a derivative of the CBP-Cre fusion protein by introduction of the ileY gene. Cultures of BL21 gold DE3 cells containing T7-driven vectors for a CBP/Cre-recombinase fusion gene with either three rare leucine (CBP-3xL-Cre, codon CUA) or isoleucine codons (CBP-3xL-Cre, codon AUA) at the 5′ end of the gene, and the indicated pACYC-based tRNA expression vectors were induced at mid-log phase for 1 h with 1 mM IPTG. Fifteen μl of whole cell lysate were loaded on a 4-20% PAGE gel and stained with Coomassie blue. The position of the induced fusion protein is indicated by an arrow. A sample of CBP-Cre without any extra codons was added as a reference. The lanes labeled with RG contain an expression vector for the tRNA genes argU and gly U. The lanes labeled RIL contain an expression vector for the argU, ileY and leuW tRNA genes.

FIG. 3 demonstrates the functionality of the proL gene in an RILP array. Cultures of BL21 goldDE3 strains containing pACYC-based vectors expressing copies of the indicated E. coli tRNA genes and the T7-driven expression vectors for human cardiac troponin T (argu-dependent) and the Cre-recombinase/CBP fusion genes CBP-exi-Cre (ileY-dependent) and CBP-3xP-Cre (proL-dependent) were induced for 2 h with 1 mM IPTG. Fifteen μl of whole cell lysate were loaded on 4-20% PAGE-gels and stained with Coomassie blue. HcTnT is an argu-dependent T7-driven recombinant human cardiac troponin T expression construct (Hu et al.). CBP-3xi-Cre and CBP-3xP-Cre are T7-driven CBP-Cre-recombinase fusion genes containing either three AUA(3xi) codons or three CCC(3xP) codons near the N-terminus of the fusion protein. Cells with the RILP array (containing the proL tRNA gene), but not the RIL array, allow efficient expression of the recombinant CBP-3xP-Cre gene. The arrows indicate the position of the recombinant gene products. RIL: argU, ileY and leu W tRNA genes. RILP: argU, ileY, leuW, and proL tRNA genes.

FIG. 4 reveals that the RIL and RILP arrays do not significantly affect the level of expression of well-expressed proteins. The indicated host cells were transformed by T7-driven expression vectors for JNK (human c-jun N-terminal kinase), λ-phosphatase or calmodulin. Cultures were induced at mid-log growth for 2 h with 1 mM IPTG. Fifteen μl of induced cultures were denatured by boiling in SDS loading buffer, separated on a 4-20% PAGE gel and stained with Coomassie blue. The positions of the induced heterologous proteins are indicated by arrows. “-”: BL21 gold DE3. RIL: BL21 gold DE3 with the argU, ileY and leuW tRNA gene array. RILP: BL21 gold DE3 with the argU, ileY, leuW and proL tRNA gene array.

FIG. 5 shows that high level expression of Pfu-polymerase depends on the presence of extra copies of both the argU and ileY genes. Cultures of BL21 gold DE3 strains containing pACYC-based vectors expressing copies of the indicated E. coli tRNA genes and the T7-driven expression vectors for Pfu-polymerase, human cardiac troponin T and CBP/Cre-recombinase were induced for 2 h with 1 mM IPTG. Fifteen μl of whole cell lysate were loaded on 4-20% PAGE-gels and stained with Coomassie blue. High level of expression of human cardiac troponin T (hcTnT) in E. coli (expressed from a pET21b construct) is dependent on removal of two tandem AGA/AGG codons or rescue by extra copies of the argU tRNA gene. CBP-3xi-Cre is a CBP-tagged Cre-recombinase construct containing 3 extra AUA codons (coding for isoleucine) at the 5′-end of the recombinant gene. High level expression of this protein in E. coli is dependent on the presence of extra copies of E. coli IleY t-RNA gene (see FIG. 2). RG: pACYC with argU and glyU tRNA genes. RI: argU and ileY tRNA genes. IL: ileY and leu W tRNA genes. RIL: argU, ileY, and leu W tRNA genes.

FIG. 6 demonstrates that failure of the RG array to support efficient Pfu DNA-polymerase expression is not due to a negative effect of the glyU gene. The experiment displayed in FIG. 6 was repeated using an argU/leuW array (RL) instead of the argU/glyU array (RG), yielding the same result. Cultures of BL21 goldDE3 strains containing pACYC-based vectors expressing copies of the indicated E. coli tRNA genes and the T7-driven expression vector for Pfu polymerase were induced for 2 h with 1 mM IPTG. Fifteen μL of whole cell lysate were loaded on 4-20% PAGE gels and stained with Coomassie stain. RL: pACYC with argU and leuW tRNA genes. RI: argU and ileY tRNA genes. IL: ileY and leuW tRNA genes. RIL: argU, ileY, and leuW tRNA genes.

FIG. 7 indicates that Pfu DNA polymerase I expression correlates with functional argU and ileY expression. Pfu DNA polymerase I was expressed in BL21 goldDE3 strains either containing no tRNA expression vector (−), pACYC-RIL, or two different isolates of the RILP expression vector. Both RILP isolates displayed functional argU expression at the same level as observed with pACYC-RIL vector. However, RILP9 displays no functional ileY expression, and in RILP 16 functional ileY expression is diminished when compared to RIL. Functional ileY expression was evaluated by rescue of the ileY-dependent production of CBP-3xi-Cre. Fifteen μl of the indicated cultures induced at mid-log growth for 2 h with 1 mM IPTG were loaded on a 4-20% PAGE gel and the separated proteins were visualized by Coomassie blue staining.

FIG. 8 demonstrates that the expression of Pfu-polymerase II subunits I and II are dependent on functional co-expression of the argu and ileY genes. Pfu DNA polymerase I and the two sub-units of Pfu-DNA polymerase II were expressed in the indicated host strains. Fifteen μl of the cultures induced at mid-log growth for 2 h with 1 mM IPTG were loaded on a 4-20% PAGE gel, and the separated proteins were visualized by Coomassie blue staining. “-”: BL21goldDE3. RG: argU and glyU tRNA genes. RI: argU and ileY tRNA genes. IL: ileY and leuW tRNA genes. RIL: argU, ileY, and leuW tRNA genes.

### DETAILED DESCRIPTION OF THE INVENTION

There is a continuing need in the art for means to improve the efficiency of expression of heterologous proteins in E. coli and other host cells. This invention provides methods and cell lines that overcome the problem of poor codon usage, which limits the expression of certain heterologous proteins, and provides for higher levels of expression of such proteins. Poor codon usage is overcome in the present invention by identifying those codons rarely used in the host cell, yet required for expression of the protein of interest. The genes for the cognate tRNA species of the expression-limiting codons are then introduced into the host cell, thereby enabling a higher level of expression of the protein, e.g., at least two-fold higher than in the absence of the tRNA gene array.

As used herein, the term “host cell” refers to a cell used to express a protein of interest by incorporating a recombinant DNA molecule which encodes the protein. The host cell can be any type of prokaryotic or eukaryotic cell suitable for the expression of recombinant proteins. Frequently, the host cell will be a bacterium, a yeast cell, an insect cell, a plant cell or a mammalian cell.

This invention is directed toward the problem of rare codon usage in a host cell, which limits expression of a desired recombinant protein. “Codon usage” refers to the frequency with which a given codon appears in the coding regions of a gene. The codon usage of a host cell refers to the average codon usage for known genes which are endogenous to the host cell. A codon or its usage is “rare” if its frequency of use in the host cell is such that depletion of the corresponding tRNA species occurs during expression (particularly high level expression (see below)) of a heterologous protein of interest. A codon which is “rare” in a given host cell may be one which is normally not used by the host cell at all or which is used by the host cell in less than 1% and even less than 0.5% of the host cell genes, or may be one which becomes limiting for the level of expression of a protein of interest. For example, several rare codons found in different organisms are presented in Tables 1-4.

A gene of interest which is introduced into a host cell is said to be “heterologous” if the frequency of codon usage for one or more codons in that gene is different from the frequency of codon usage for those codons in the host cell. For example, the gene of interest can have a different frequency of codon usage for one or several codons because the gene was isolated from another organism, which utilizes certain codons at a different frequency than the host cell. The organism from which a heterologous gene is obtained for use in the invention may be any organism. For example, the organism can be a prokaryote or eukaryote; it can be a bacterium, a yeast, a plant, an animal, or a mammal. A gene whose codon usage differs significantly from that of the host cell is said to have “biased codon usage.” High level expression of a protein having biased codon usage can cause depletion of one or more tRNA species.

“High level expression” is the expression of a recombinant protein in a host cell in amounts higher than would be typical for an endogenous protein of the host cell. High level expression can result in the recombinant protein comprising from about 0.5%, 1%, 5%, 10% and even up to about 30% or more of total cell protein. High level expression is usually accomplished by introduction of the gene encoding the recombinant protein incorporated within an expression vector. An inducer can be added to the culture medium so as to activate the expression of the recombinant protein. For example, if the expression vector contains the promoter for T7 bacteriophage RNA polymerase, and the host cell chromosome contains the gene for T7 RNA polymerase under control of the lac repressor, then induction can be obtained by adding isopropylthio-β-galactoside (IPTG) to the medium. Suitable expression vectors are known in the art which, together with the appropriate host cell, will yield inducible, high level expression of a recombinant protein.

Any protein of interest can be employed in the invention, provided that it can be expressed from a recombinant DNA molecule in a suitable host cell. Preferably, the protein is one whose expression is limited by poor codon usage in the host cell. More preferably, a host cell is capable of high level expression of a protein of interest once the appropriate tRNA genes have been introduced into the host cell. Examples of proteins that are suitable for use with the invention are chicken histone H5 (Gerchman, S. E., Graziano, V., Ramakrishnan, V., “Expression of chicken linker histones in E. coli: Sources of problems and methods for overcoming some of the difficulties,” Protein Express. Purif. 5:242-251 (1994), bovine placental lactogen (Kane, J. F., Violand, B. N., Curran, D. F., Staten, N. R., Duffin, K. L., Bogosian, G., “Novel in-frame two codon translational hop during synthesis of bovine placental lactogen in a recombinant strain of Escherichia coli,” Nucleic Acids Res. 20:6707-6712 (1992), human tropoelastin (Martin, S. L., Vrhovski, B., Weiss, A. S., “Total synthesis and expression in Escherichia coli of a gene encoding human tropoelastin,” Gene 154:159-166 (1995), human granulocyte-macrophage colony stimulating factor (Hua, Z., Wang. H., Chen, D., Chen, Y., Zhu, D., “Enhancement of expression of human ganulocyte-macrophage colony stimulating factor by argU gene product in Escherichia coli,” Biochem. Mol. Biol. Int. 32:537-543 (1994)), human catechol-O-methyltransferase, as well as archebacterial proteins such as Pyrococcus furiosus DNA polymerases I and II.

1. Arrays of tRNA Genes Useful According to the Invention

The invention calls for introduction of an “array” of tRNA genes into the host cell. An array of tRNA genes consists of preferably 3 or more, e.g., 4, 5, 6, 7, 8, 9, or 10 tRNA genes which have been introduced into one or more vectors by cloning. As used herein, a “set” of tRNA genes contains only two tRNA genes; specific sets of tRNA genes useful according to the invention are described herein. A set, as used herein, does not consist of the pair of tRNA genes, E. coli genes argU and ileX.

According to one embodiment, an array of tRNA genes is introduced on a single vector. According to another embodiment, two or more vectors are introduced into the host cell, each containing either a single tRNA gene or an array of tRNA genes. In yet another embodiment, 2, 3, 4, 5, 6, 7, 8, 9, or 10 individual tRNA genes or one or more arrays of tRNA genes are inserted into the chromosome of the host cell, resulting in functional expression of the tRNA genes. In any embodiment of this invention, the tRNA genes introduced into the host cell may be different genes or multiple copies of identical tRNA genes. The vectors should be replication competent, suitable for transformation or transfection of the host cell, and should result in the functional expression of the tRNA molecules encoded by the genes.

The tRNA genes are functionally expressed if the host cell produces the corresponding functional tRNA molecules, which can be used in the translation of proteins in the host cell. The expression of the tRNA genes can be regulated by the endogenous promoter for each gene, or a different promoter or promoters can be added to control the transcription of any combination of the genes, including each gene individually or all of the genes simultaneously, under the control of a single promoter. The expression of two or more tRNA genes in the same host cell at the same time is referred to as “co-expression.” Co-expression can be achieved, for example, by linking two or more tRNA genes to control by a single promoter.

The reported effects of rare codons generally refer to one codon occurring consecutively or in clusters. Reported experimental approaches have used either synonymous replacement of the codons or expression of a specific tRNA species along with a gene of interest. However, co-expression of an array of tRNA genes corresponding to rare codons according to the invention is contemplated to have a correspondingly greater effect on protein expression by permitting higher levels of expression of a selected protein than in cells lacking the tRNA gene array. Hence, expression of proteins containing multiple rare codons may be efficiently salvaged by simultaneous expression of their collective cognate tRNA genes.

The inventive approach to compensate for multiple rare codons is to generate a vector that co-expresses preferably three or more tRNA genes at the same time. Simultaneous expression of several tRNA genes from a vector also will generate a generic host for expression of genes from a variety of species with different codon biases. In a preferred embodiment, the vector is a low copy number vector. A low copy number vector is one having on average between about 1 and about 10 copies per cell. A medium copy number vector is one having on average between about 11 and about 50 copies per cell. A high copy number vector is one having on average between about 51 and about 200 copies per cell.

In one embodiment of the invention, the vector contains one or more transcription control elements in addition to the array of tRNA genes. A transcription control element is a nucleotide sequence that modifies, i.e., enhances or limits, transcription of the tRNA genes. A transcription control element can be, for example, a promoter or a terminator. A vector of the invention can also contain additional sequences, e.g., an origin of replication, a drug resistance gene, or a reporter gene, as required for its replication, function, or detection in the host cell, or as required for selection of transformed or transfected host cells.

A vector of the invention is selected for suitability of use with the chosen host strain. Regardless of the host cell type, the vector containing the tRNA gene array is preferably a low to medium copy number expression vector. The following are examples. Bacterial species that are suitable for use with the invention include Escherichia coli and Bacillus subtilis. Yeast species that are suitable for use with the invention include Saccharomyces cerevisiae and Pichia pastoris.

Specialized arrays of tRNA genes biased towards the codon usage of specific organisms are utilized according to the invention. Table 1 lists the codons that are used with a frequency of less than 1% in E. coli, and compares the frequency of use in several other organisms. Frequently used codons (arbitrarily designated as codons with a frequency of more than 1.5%) in heterologous genes constitute potential limitations to heterologous protein expression in E. coli. Using Table 1, or analogous tables prepared for other host cells, arrays of tRNA genes can be designed to meet the needs of a high level expression system for any organism. For example, one possible array for expression of human proteins in E. coli would include the cognate tRNA genes for codons AGG and AGA (Arg); CCC, CCU, and CCA (Pro); GGA and GGG (Gly); and UCC (Ser). Another example of an array that could be selected from Table 1 is the combination of arginine and proline codons (AGG and AGA (Arg); CCC (Pro)), which could be provided, for example, by the E. coli tRNA genes argU and proL. Yet another example of an array that could be selected from Table 1 is the combination of arginine, isoleucine, and leucine codons (AGG and AGA (Arg); AUA (Ile); and CUA (Leu)), provided, for example, by the E. coli tRNA genes argU, ileY, and leu W. By designing arrays of tRNA genes in this way, it is possible to tailor an expression host to meet the needs of any desired recombinant protein with a particular codon bias.

In one embodiment of the invention, the host cell and a vector containing three or more tRNA genes are made available as a kit. The vector of such a kit contains an array comprising preferably 3, 4, 5, 6, 7, 8, 9, or 10 tRNA genes tailored to the codon use frequency of, e.g., the proteins of a particular species and a particular host cell. In an alternative embodiment, the kit contains two or more vectors, each containing one or more tRNA genes or an array of tRNA genes. The user of the kit can choose particular tRNA genes to add to the array as needed for the production of a given heterologous protein with biased codon usage. For example, the vector of the kit can include a restriction endonuclease cleavage site, such that the user can cleave the vector, add two or more tRNA genes, and ligate the vector using a DNA ligase to form a new vector. The vector supplied with the kit is suitable for use with the host cell supplied with the kit. The kit also will include packaging materials for the vector and/or host cell.

Particularly useful sets of tRNA genes where only two tRNA genes are present in the set are as follows. In one such embodiment of this invention is a set containing two tRNA genes directed to the codons AGA or AGG (Arg) and AUA (Ile).

In a preferred embodiment of an array according to the invention, which targets the codons AGA or AGG (Arg)), CUA (Leu), AUA (Ile) and CCC (Pro) in E. coli, the choice of these codons is based on their infrequent use in E. coli (see Table 1), and the availability of the cognate tRNA genes. To prepare this embodiment, an array of four tRNA genes which encode tRNAs rarely expressed in E. coli, argU, ileY, leuW and proL (recognizing the codons AGA/AGG, AUA, CUA and CCC, respectively) was introduced into a low copy number plasmid, pACYC-LIC (see Example 1). These four genes, which are rare in E. coli, were isolated from E. coli K12. Introduction of this plasmid into suitable protein expression hosts such as E. coli BL21 DE3 allows high level of expression of proteins normally restricted by the presence of rare codons.

Another embodiment of this invention is a method of producing a protein of interest using a host cell and one or mre suitable vectors containing an array of tRNA genes. The gene for the protein of interest is introduced into the host cell or is present in the host cell. The gene can be introduced by any method known in the art. For example, the gene can be introduced by cotransforming or cotransfecting the host cell with an additional vector or nucleic acid construct which contains the gene and any nucleotide sequences necessary or desired to control transcription of the gene.

In a preferred embodiment, the method is used to produce the protein of interest, which is then isolated from the host cell and purified. Methods of protein purification are well known in the art and must be adapted to the requirements of each individual protein. The method applied to a given recombinant protein will depend on factors such as the size, charge, and hydrophobicity of the protein and whether the protein is secreted from the host cell, contained within the cytoplasm or within membranes, or precipitated in the form of inclusion bodies. See generally, Deutscher, M. P., “Guide to Protein Purification,” Methods Enzymol., Academic Press, Vol. 182 (1990); Le, H. V., Trotta, P. P., “Purification of secreted recombinant proteins from Escherichia coli,” Bioprocess Technol. 12:163-181 (1991); and Ladisch, M. R., Kohlmann, K. L., “Recombinant Human Insulin,” Biotechnol. Prog. 8:469-478 (1992).

2. Host Cells Useful According to the Invention

2a. Bacterial Host Cells Useful According to the Invention

Suitable host cells for transfection with a set or array of tRNA genes and expressing the DNA encoding the desired polypeptide are the prokaryotic, yeast, or higher eukaryote cells. Suitable prokaryotes for this purpose include bacteria such as archaebacteria and eubacteria. Preferred bacteria are eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella; Bacilli such as B. subtilis and B. licheniformis (e.g., B. licheniformis 41P disclosed in DD 266,710 published Apr. 12, 1989); Pseudomonas such as P. aeruginosa, Streptomyces; Azotobacter; Rhizobia; Vitreoscilla; and Paracoccus. Suitable E. coli hosts include E. coli W3110 (ATCC 27,325), E. coli 294 (ATCC 31,446), E. coli B, and E. coli X1776 (ATCC 31,537). These examples are illustrative rather than limiting.

Mutant cells of any of the above-mentioned bacteria may also be employed.

It is, of course, necessary to select the appropriate bacteria taking into consideration replicability of the replicon in the cells of a bacterium. For example, E. coli, Serratia, or Salmonella species can be suitably used as the host when well known plasmids such as Col E1, pACYC and RKZ-based vectors are used to supply the replicon. E. coli strain W3110 is a preferred host or parent host because it is a common host strain for recombinant DNA product fermentations. Preferably, the host cell should secrete minimal amounts of proteolytic enzymes or should be protease deficient. Cells which are lacking or deficient in Lon and OmpT proteases are preferred. For example, strain W3110 may be modified to effect a genetic mutation in the genes encoding proteins, with examples of such hosts including E. coli W3110 strain 27C7. The complete genotype of 27C7 is tonA.DELTA.ptr3 phoA.DELTA.E15.DELTA.(argF-lac) 169 ompT.DELTA. degP41kan.sup.r. Strain 27C7 was deposited on Oct. 30, 1991 in the American Type Culture Collection as ATCC No. 55,244. Alternatively, the strain of E. coli having mutant periplasmic protease disclosed in U.S. Pat. No. 4,946,783 issued Aug. 7, 1990 may be employed. Alternatively, in vitro methods of cloning, e.g., PCR or other nucleic acid polymerase reactions, are suitable. E. coli BL21 also is useful according to the invention (Studier et al., 1986, J. Mol. Biol., 189:113; Studier et al., 1990, Methods Enzy. 185:60). Another preferred attribute of the host cell is the Hte (high transformation efficiency) phenotype, which increases the the transformation efficiency of large and ligated DNA. RecA positive strains allow faster growth and yield high levels of protein expression. Endonuclease I (e.g., EndA 1) deficient strains yield miniprep DNA that is less prone to degradation.

For example, strain W3110 may be modified to effect a genetic mutation in

the genes encoding proteins endogenous to the host, with examples of such hosts including E. coli W3110 strain 1 A2, which has the complete genotype tonA.DELTA.; E. coli W3110 strain 9E4, which has the complete genotype tonA.DELTA. ptr3; E. coli W3110 strain 27C7 (ATCC 55,244), which has the complete genotype tonA.DELTA. ptr3 phoA.DELTA.E15 (argF-lac)169 ompT.DELTA. degP41kan.sup.r; E. coli W3110 strain 37D6, which has the complete genotype tonA.DELTA. ptr3 phoA.DELTA.E15.DELTA.(argF-lac)169 ompT.DELTA. degP41kan.sup.r rbs7.DELTA. ilvG; E. coli W3110 strain 40B4, which is strain 37D6 with a non-kanamycin resistant degP deletion mutation; and an E. coli strain having mutant periplasmic protease disclosed in U.S. Pat. No. 4,946,783 issued Aug. 7, 1990.

2b. Fungi, Plant and Insect Host Cells Useful According to the Invention

In addition to prokaryotes, eukaryotic microbes such as filamentous fungi or yeast are suitable cloning or expression hosts for set or arrays of tRNA genes and polypeptide-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe (Beach and Nurse, Nature, 290: 140 (1981); EP 139,383 published May 2, 1985); Kluyveromyces hosts (U.S. Pat. No. 4,943,529; Fleer et al., supra) such as, e.g., K lactis

(MW98-8C, CBS683, CBS4574; Louvencourt et al., J. Bacteriol., 737 (1983)), K fragilis (ATCC 12,424), K bulgaricus (ATCC 16,045), K wickeramii (ATCC 24,178), K waltii (ATCC 56,500), K drosophilarum (ATCC 36,906; Van den Berg et al., supra), K. thermotolerans, and K marxianus; yarrowia (EP 402,226); Pichia pastoris (EP 183,070; Sreekrishna et al., J. Basic Microbiol., 28: 265-278 (1988)); Candida; Trichoderma reesia (EP 244,234); Neurospora crassa (Case et al., Proc. Natl. Acad. Sci. USA, 76: 5259-5263 (1979)); Schwanniomyces such as Schwanniomyces occidentalis (EP 394,538 published Oct. 31, 1990); and filamentous fungi such as, e.g., Neurospora, Penicillium, Tolypocladium (WO 91/00357 published Jan. 10, 1991), and Aspergillus hosts such as A. nidulans (Ballance et al., Biochem. Biophys. Res. Commun., 112: 284-289 (1983); Tilburn et al., Gene, 26: 205-221 (1983); Yelton et al., Proc. Natl. Acad. Sci. USA, 81: 1470-1474 (1984)) and A. niger (Kelly and Hynes, EMBO J, 4: 475-479 (1985)).

Suitable host cells according to the invention and appropriate for the expression of the DNA encoding the desired polypeptide may also be derived from multicellular organisms. Such host cells are capable of complex processing and glycosylation activities. In principle, any higher eukaryotic cell culture is suitable, whether from vertebrate or invertebrate culture. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. See, e.g., Luckow et al., Bio/Technology, 6: 47-55 (1988); Miller et al., in Genetic Engineering, Setlow, J. K. et al., eds., Vol. 8 (Plenum Publishing, 1986), pp. 277-279; and Maeda et al., Nature, 315: 592-594 (1985). A variety of viral strains for transfection are publicly available, e.g., the L-1 variant of Autographa californica NPV and the Bm-5 strain of Bombyx mori NPV, and such viruses may be used as the vector herein according to the present invention, particularly for transfection of Spodoptera frugiperda cells.

Plant cell cultures of cotton, corn, potato, soybean, petunia, tomato, and tobacco can be utilized as hosts. Typically, plant cells are transfected by incubation with certain strains of the bacterium Agrobacterium tumefaciens, which has been previously manipulated to contain the DNA encoding the desired polypeptide. During incubation of the plant cell culture with A. tumefaciens, the DNA encoding the desired polypeptide is transferred to the plant cell host such that it is transfected, and will, under appropriate conditions, express the DNA encoding the desired polypeptide. In addition, regulatory and signal sequences compatible with plant cells are available, such as the nopaline synthase promoter and polyadenylation signal sequences (Depicker et al., J. Mol. Appl. Gen., 1:561, (1982)). In addition, DNA segments isolated from the upstream region of the T-DNA 780 gene are capable of activating or increasing transcription levels of plant-expressible genes in recombinant DNA-containing plant tissue. EP 321,196 published Jun. 21, 1989.

2c. Mammalian Host Cells Useful According to the Invention

Examples of useful mammalian host cell lines are monkey kidney Cvl line transformed by SV40 (COS-7, ATCC CRL 1651); human embryonic kidney line (293 or 293 cells subcloned for growth in suspension culture, Graham et al., J. Gen Virol., 36: 59 (1977)); baby hamster kidney cells (BHK, ATCC CCL 10); Chinese hamster ovary cells/-DHFR(CHO, Urlaub and Chasin, Proc. Natl. Acad. Sci. USA, 77: 4216 (1980)); mouse sertoli cells (TM4, Mather, Biol. Reprod., 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383: 44-68 (1982)); MRC 5 cells; FS4 cells; and a human hepatoma line (Hep G2).

Host cells are transfected and preferably transformed with the above-described set or array of tRNA genes and expression or cloning vectors of this invention and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO4 and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in section 1.82 of Sambrook et al., Molecular Cloning: A Laboratory Manual (New York: Cold Spring Harbor Laboratory Press, 1989), or electroporation is generally used for prokaryotes or other cells that contain substantial cell-wall barriers. Infection with Agrobacterium tumefaciens is used for transformation of certain plant cells, as described by Shaw et al., Gene, 23: 315 (1983) and WO 89/05859 published Jun. 29, 1989. In addition, plants may be transformed using ultrasound treatment as described in WO 91/00358 published Jan. 10, 1991.

For mammalian cells without such cell walls, the calcium phosphate precipitation method of Graham and van der Eb, Virology, 52: 456-457 (1978) is preferred. General aspects of mammalian cell host system transformations have been described by Axel in U.S. Pat. No. 4,399,216 issued Aug. 16, 1983. Transformations into yeast are typically carried out according to the method of Van Solingen et al., J. Bact., 130: 946 (1977) or Hsiao et al., Proc. Natl. Acad. Sci. USA, 76: 3829 (1979). However, other methods for introducing DNA into cells, such as by nuclear microinjection, electroporation, bacterial protoplast fusion with intact cells, or polycations, e.g., polybrene, polyornithine, etc., may also be used. For various techniques for transforming mammalian cells, see Keown et al., Methods in Enzymology (1989), Keown et al., Methods in Enzymology (1990) Vol. 185, pp. 527-537, and Mansour et al., Nature, 336: 348-352 (1988).

If prokaryotic cells are used to produce the polypeptide of interest in accordance with the method of this invention, they are cultured in suitable media in which the promoter can be constitutively or artificially induced as described generally, e.g., in Sambrook et al., supra, 1989.

Any necessary supplements besides carbon, nitrogen, and inorganic phosphate sources may also be included at appropriate concentrations introduced alone or as a mixture with another supplement or medium such as a complex nitrogen source. The pH of the medium may be any pH from about 5 to 9, depending mainly on the host organism.

If mammalian host cells are used to produce the polypeptide of this invention, they may be cultured in a variety of media. Commercially available media such as Ham's F10 (Sigma), Minimal Essential Medium ((MEM), Sigma), RPMI-1640 (Sigma), and Dulbecco's Modified Eagle's Medium (DMEM), Sigma) are suitable for culturing the host cells. In addition, any of the media described in Ham and Wallace, Meth. Enz., 58: 44 (1979), Barnes and Sato, Anal. Biochem., 102: 255 (1980), U.S. Pat. Nos. 4,767,704; 4,657,866; 4,927,762; or 4,560,655; WO 90/03430; WO 87/00195; U.S. Pat. Re. No. 30,985; or U.S. Pat. No. 5,122,469, the disclosures of all of which are incorporated herein by reference, may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin TM. drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, or can be modified as needed to obtain optimal expression, and will be apparent to the ordinarily skilled artisan. In general, principles, protocols, and practical techniques for maximizing the productivity of in vitro mammalian cell cultures can be found in Mammalian Cell Biotechnology: A Practical Approach, M. Butler, ed., (IRL Press at Oxford University Press, Oxford, 1991).

3. Purification of a Polypeptide of Interest From a Host Cell

It is often preferred to purify the polypeptide of interest from recombinant cell proteins or polypeptides to obtain preparations that are substantially homogeneous as to the polypeptide of interest. In one embodiment, as a first step, the culture medium or lysate is centrifuged to remove particulate cell debris. The membrane and soluble protein fractions may then be separated if necessary. The polypeptide may then be purified from the soluble protein fraction and from the membrane fraction of the culture lysate, depending on whether the polypeptide is membrane bound, is soluble, or is present in an aggregated form. The polypeptide thereafter is solubilized and then subsequently refolded if necessary using an appropriate buffer.

Insoluble, non-native polypeptide is isolated from the prokaryotic host cells in a suitable isolation buffer by any appropriate technique, e.g., one involving exposing the cells to a buffer of suitable ionic strength to solubilize most host proteins, but in which aggregated polypeptide is substantially insoluble, disrupting the cells so as to release the inclusion bodies and make them available for recovery by, for example, centrifugation. This technique is well known, and is described, for example, in U.S. Pat. No. 4,511,503.

Briefly, the cells are suspended in the buffer (typically at pH 5 to 9, preferably about 6 to 8, using an ionic strength of about 0.01 to 2M, preferably 0.1 to 0.2M). Any suitable salt, including sodium chloride, is useful to maintain a sufficient ionic strength value. The cells, while suspended in this buffer, are then disrupted by lysis using techniques commonly employed such as mechanical methods (e.g., a Manton-Gaulin press, a French press, or a sonic oscillator), or by chemical or enzymatic methods.

Examples of chemical or enzymatic methods of cell disruption include spheroplasting, which entails the use of lysozyme to lyse the bacterial wall (Neu et al., Biochem. Biophys. Res. Comm., 17: 215 (1964)), and osmotic shock, which involves treatment of viable cells with a solution of high tonicity and with a cold-water wash of low tonicity to release the polypeptides (Neu et al., J. Biol. Chem., 240: 3685-3692 (1965)). A third method, described in U.S. Pat. No. 4,680,262 issued Jul. 14, 1987, involves contacting the transformed bacterial cells with an effective amount of a lower alkanol having 2 to 4 carbon atoms for a time and at a temperature sufficient to kill and lyse the cells.

After the cells are disrupted, the suspension is typically centrifuged to pellet the inclusion bodies. In one embodiment, this step is carried out at about 500 to 15,000×g, preferably about 12,000×g, in a standard centrifuge for a sufficient time that depends on volume and centrifuge design, usually about 10 minutes to 0.5 hours. The resulting pellet contains substantially all of the insoluble polypeptide fraction, but if the cell disruption process is not complete, it may also contain intact cells or broken cell fragments. Completeness of cell disruption can be assayed by resuspending the pellet in a small amount of the same buffer solution and examining the suspension with a phase contrast microscope. The presence of broken cell fragments or whole cells indicates that additional disruption is necessary to remove the fragments or cells and the associated non-refractile polypeptides. After such further disruption, if required, the suspension is again centrifuged and the pellet recovered, resuspended, and analyzed. The process is repeated until visual examination reveals the absence of broken cell fragments in the pelleted material or until further treatment fails to reduce the size of the resulting pellet.

In an alternative embodiment, the polypeptide of interest, preferably exogenous, is isolated by solubilization in a suitable buffer. This procedure can be in situ solubilization involving direct addition of reagents to the fermentation vessel after the polypeptide has been produced recombinantly, thereby avoiding extra steps of harvesting, homogenization, and centrifugation to obtain the polypeptide. The remaining particulates can be removed by centrifugation or filtration, or combinations thereof. Alternatively, and more preferably, one may use a multiple-phase isolation/extraction system for purifying polypeptides from the remaining particulates.

The above disclosure generally describes the present invention. A more complete understanding can be obtained by reference to the following specific examples, which are provided herein for purposes of illustration only and are not intended to limit the scope of the invention.

### EXAMPLE 1

Construction of Expression Vectors Containing E. coli tRNA Gene Sets or Arrays.

Expression vectors for tRNA gene sets or arrays, with the exception of pACYC-RG, were constructed by inserting PCR-amplified tRNA gene sets or arrays containing SpeI and XhoI sites at their 5′ and 3′ end between the SpeI and XhoI sites of the pACYC 184 derivative pACYC-LIC. pACYC-LIC contains a multiple cloning site including LIC-sites replacing most of the tet gene of pACYC 184. The tet promoter is retained and drives expression of inserts within the polylinker.

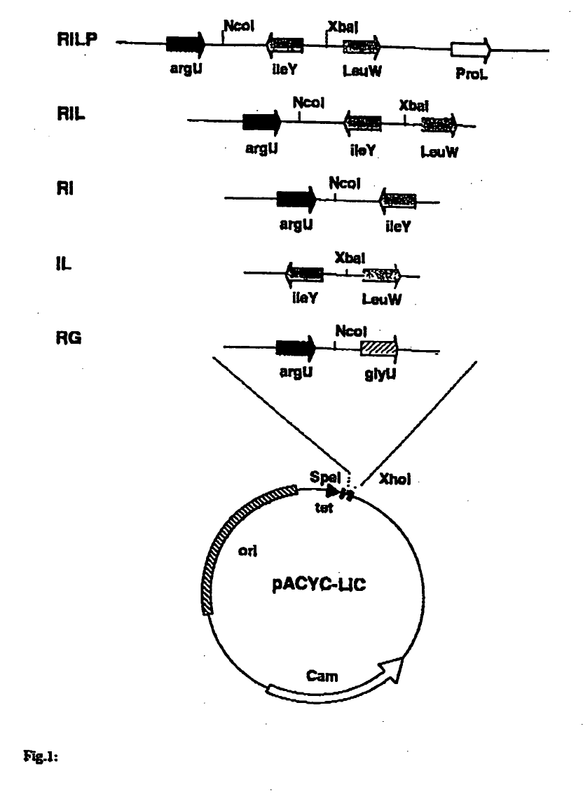

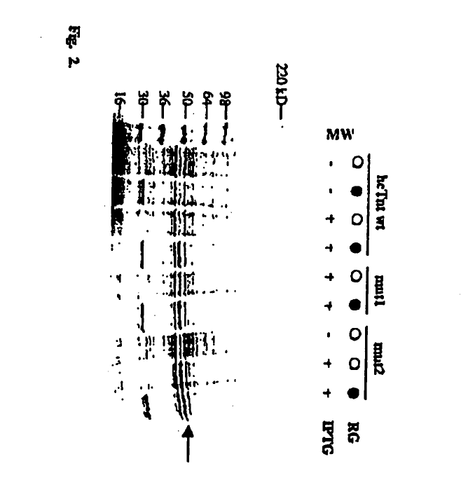

The RG-set was cloned into pACYC-LIC by ligation-independent cloning. The sets were generated by first amplifying the individual tRNA genes with primers including recognition sites for restriction enzymes, ligating the cut tRNA fragments to each other and then amplifying the desired sets or arrays with flanking primers using the ligation reaction as the template. The RILP array was generated by first ligating the proL gene fragment to the RIL array and then amplifying the whole array by using the flanking primer as described for the other arrays. For an overview of the constructs see FIG. 1. Hereinafter, R will refer to the argU tRNA gene, I to the ileY tRNA gene, L to the leu W tRNA gene, P to the tRNA gene and G to the glyU tRNA gene when used in the context of tRNA gene sets or arrays or the derived vectors.

The individual tRNA genes were isolated by PCR using E. coli K12 DNA as a template. ArgU was amplified using the primer GAC ACT AGT AAT CAG ACG CGG TCG TTC AC (SEQ ID NO:1; for RI, RIL and RILP; SpeI site underlined) or GAC GAC GAC AAG AAT CAG ACG CGG TCG TTC AC (SEQ ID NO:2; for RG; LIC site underlined) as forward primer and CTG CCA TGG TGG AGG ATA TAA AGA AGG CG (SEQ ID NO:3; NcoI site underlined) as the reverse primer. The primers anneal at bp 8041 (forward) and bp 8260 (reverse) in the Genbank file Accession Number AE000159. The amplified fragment is 220 bp long and contains 104 bp 5′ and 38 bp 3′ to the argU tRNA gene. Extensions containing recognition sites for SpeI (forward primer) and NcoI restriction endonucleases were added for construction purposes. The ileY tRNA gene was amplified using the primer CAG CCA TGG CCT TGA AAT GGC GTT AGT CA (SEQ ID NO:4; for RI and RIL; NcoI site underlined) or GAC ACT AGT CCT TGA AAT GGC GTT AGT CA (SEQ ID NO:5; for IL; SpeI site underlined) as forward primer and CAG TCT AGA TCA TCA TGT TTA TTG CGT GG (SEQ ID NO:6; for IL and RIL; XbaI site underlined) or GAC CTC GAG TCA TCA TGT TTA TTG CGT GG (SEQ ID NO:7; for RI; XhoI site underlined) as the reverse primer. The primers anneal at bp 7741 and bp 7950 in the Genbank file Accession Number AE000350. The amplified fragment is 210 bp long and contains 92 bp 5′ and 54 bp 3′ to the IleY tRNA gene. Extensions containing recognition sites for NcoI or SpeI (forward primer) and XbaI or XhoI (reverse primer) restriction endonucleases were added for construction purposes. The leuW tRNA gene was amplified using the primer CAG TCT AGA GAA TCC CGT CGT AGC CAC CA (SEQ ID NO:8; XbaI site underlined) as forward primer and GAC CTC GAG GGC ATC CGA TCA ACG CTT TCT (SEQ ID NO:9; XhoI site underlined) as the reverse primer. The primers anneal at bp 241 (forward) and bp 378 (reverse) in the Genbank file Accession Number J01713. The amplified fragment is 138 bp long and contains 29 bp 5′ and 33 bp 3′ to the LeuW tRNA gene. Extensions containing recognition sites for XbaI (forward primer) and XhoI restriction endonucleases were added for construction purposes. The proL tRNA gene was amplified using the primer GAC GTC GAC GTG CTG ACA GAC GAG AAG CG (SEQ ID NO:10; SalI site underlined) as forward primer and GAC CTC GAG GGT GTG GTC TGG ACG TTC TG (SEQ ID NO:11) as reverse primer. The amplified product is 310 bp long and contains 110 bp 5′ and 117 bp 3′ to the tRNA gene. The SalI and XhoI sites were included for construction purposes. The glyU tRNA gene was amplified using the primer CTG CCA TGG GGC ACT TGC TAA GGA GAG CG (SEQ ID NO:12; NcoI site underlined) as forward primer and GGA ACA AGA GGG CGT GTT TTC CTG GGT TGT TAC (SEQ ID NO:13; LIC site underlined) as the reverse primer. The amplified fragment is 209 bp long and contains 49 bp 5′ and 86 bp 3′ to the tRNA gene. All PCR reactions were carried out for 30 cycles of 95° C., 1 min, 55° C., 1 min and 72° C. for 1 min using cloned Pfu-polymerase supplemented with PEF, except for proL, which was amplified using Taq Plus precision (Stratagene).

For amplification of the sets or arrays the respective PCR products were isolated from agarose gels and digested with the appropriate restriction enzymes. The restriction digested PCR fragments were then ligated to each other for 4 h at 16° C. using T4 DNA ligase. One μl of this ligation was used as a template in a PCR reaction using the forward primer of the first tRNA gene and reverse primer of the last tRNA gene of the desired set or array. The conditions for the PCR reactions were the same as described above except that only 25 cycles were performed. The reaction product of the anticipated size for the correct product was isolated from an agarose gel, digested with SpeI and XhoI and ligated into SpeI and XhoI-digested pACYCI84-LIC. The map of the resulting plasmids is shown in FIG. 1.

### EXAMPLE 2

Test of Functional Expression of the argU, ileY, and proL tRNA Genes.

The tRNA genes in the vector were arranged in an operon-like structure driven by the promoter of the tetracycline resistance gene (FIG. 1). Some of the fragments may have contained their endogenous promoter. The fragment containing the argU gene contained its own promoter (Saxena and Walker, “Expression of argu, the Escherichia coli gene coding for a rare arginine tRNA,” J. Bacteriol. 174:1956-64 (1992)), but lacked the rho-dependent transcriptional terminators (Garcia, G. M., Mar, P. K., Mullin, D. A., Walker, J. R., Prather, N. E., “The E. coli dnaY gene encodes an arginine transfer RNA,” Cell 45:453-459 (1986)). The affected sequence was not part of the mature tRNA. The mutations may be significant for expression, however, since they disrupt a dyad symmetry element that previously has been demonstrated to suppress transcription of the argU promoter (Saxena and Walker, 1992, supra). The promoter regions for the ileY and proL genes are not defined. However, based on their natural arrangement as isolated tRNA genes within the E. coli genome the two fragments utilized can be expected to contain their own promoters. In its natural arrangement the leu W gene is part of a tRNA operon, and the amplified fragment may therefore lack an individual promoter (Nakajima, N., Ozeki, H., Shimura, Y., “Organization and structure of an E. coli tRNA operon containing seven tRNA genes,” Cell 23:239-49 (1981)).

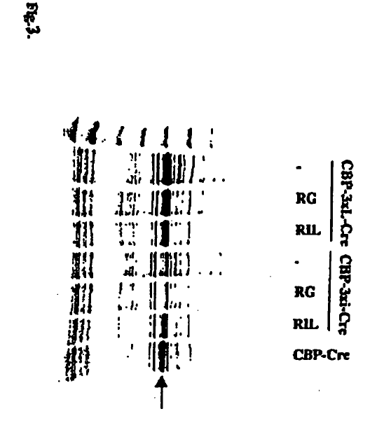

Since genes affected by AUA (ile), CGA (leu) or CCC (pro) were not available, tester constructs were generated based on the observation (Rosenberg, 1993, supra) that rare codons affect translation in E. coli most when present in a consecutive arrangement at the N-terminus of the protein. Three consecutive leucine (CUA), isoleucine (AUA), or proline (CCC) codons were introduced at the 5′ end of the recombinant CBP/Cre fusion gene. Upon IPTG induction, unmodified CBP/Cre fusion protein was expressed at approximately 30% of the total protein, most, if not all of it being soluble. As can be seen in FIG. 2, introduction of the rare leucine codon CUA did not affect expression of the recombinant gene. The functional expression of the leu W gene could therefore not be assessed. In contrast, introduction of the isoleucine codon strongly reduced expression of the recombinant protein. Expression could be rescued by expression of sets or arrays containing the ileY gene, but not by the RG (argu and glyU) set, which lacks the ileY gene. Thus, the ileY gene in the RIL array is functional and specifically rescues expression of genes affected by the rare isoleucine codon AUA.

The same strategy was applied to test for functional expression of the proL gene in the RILP array. As can be seen in FIG. 3, the presence of the RILP array, but not the RIL array, rescued expression of a tester construct containing the cognate CCC codon. Therefore, the proL gene in the RILP array is functional. However, although the argU gene in the RILP array performs at the same level as observed in the RIL array, functional expression of the ileY gene in the RILP array was diminished when compared to the RIL array. Five independent isolates of the pACYC-RILP construct were tested and the same effect was observed in all constructs, albeit to a different extent (ranging from undetectable expression to detectable, but diminished). Sequencing the RILP-tRNA gene arrays revealed that in isolates failing to show ileY activity, the ileY gene contains a point mutation (A-->T at nt 50 of the mature tRNA) that prevents proper folding of the tRNA and thus is likely to inactivate it. However, in the RILP isolates displaying diminished activity, the sequences of the argU, ileY, and leuW tRNA genes were indistinguishable from the RIL array. This observation suggests a potential incompatibility of simultaneous ectopic expression of the proL and ileY tRNA genes, possibly due to either interference of the proL gene with ileY transcription and/or processing or due to an attenuation of the host cells.

### EXAMPLE 3

Effect of RIL and RILP Arrays on Expression of Well-Expressed Genes.

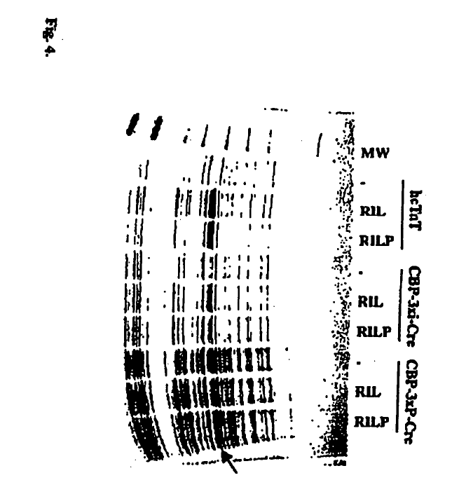

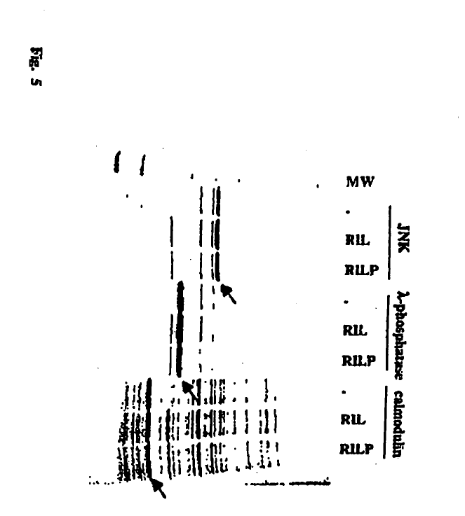

In order to test for potential deleterious effects of the RIL and RILP arrays, the expression of four well-expressing T7-driven recombinant genes were compared in BL21 goldDE3 cells and their pACYC-RIL and pACYC-RILP containing derivatives. As shown in FIG. 4, no differences were observed between BL21 goldDE3 cells and their tRNA-supplemented derivatives in the induced expression level of λ-phosphatase or JNK. However, a loss of expression was observed for calmodulin. The differences between the lines for calmodulin expression were significantly smaller when the induction time was extended, and great variations in induction efficiencies were observed for calmodulin but not for any of the other tested constructs. The reason for the suppression of calmodulin expression is unclear. For chemically competent pACYC-RIL cells, transformation efficiencies of 1×10⁸/μg of pUC18 could be achieved, which was the expected efficiency for a BL21 goldDE3 derived cell line. Aside from the induction of calmodulin, no negative effects of the pACYC-based tRNA expression arrays on host cell performance have been detected.

### EXAMPLE 4

Enhancement of Expression of Archebacterial Genes.

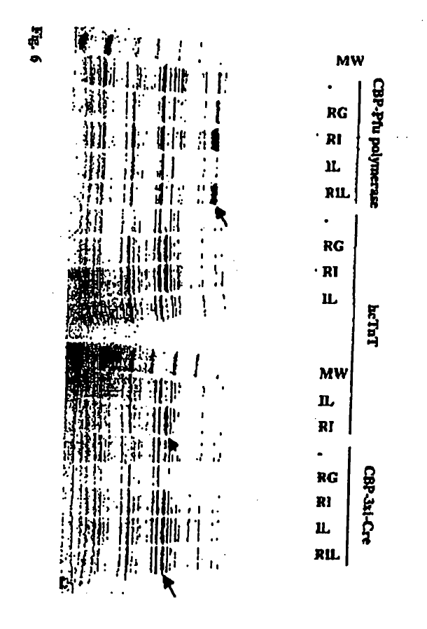

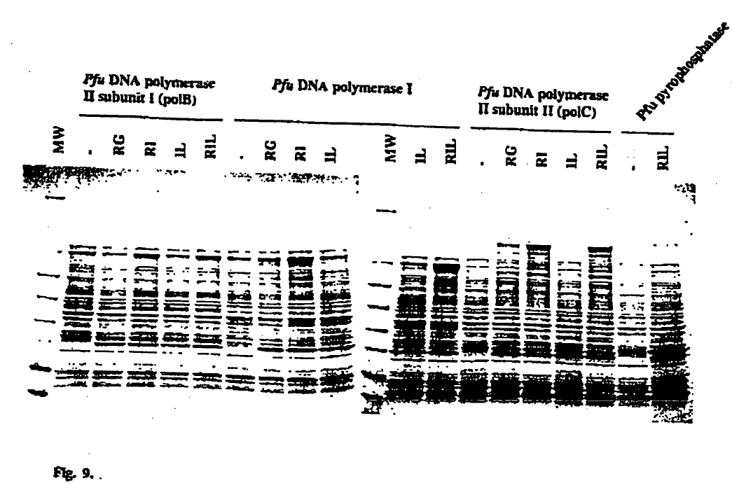

In order to find further examples of genes aided in their bacterial expression by ectopic tRNA expression, six recombinant Pyrococcus furiosus genes were tested that are only expressed poorly in E. coli. The choice of archaebacterial genes was based on their strong bias for rare E. coli codons, especially AGG/AGA and AUA (Table 1; in fact, 95% of the arginine codons are AGG or AGA). Of six tested constructs, four were rescued by co-expression of the pACYC-RIL construct. The enhanced expression of Pfu-polymerase I, Pfu polymerase II subunit I, Pfu polymerase II subunit II, and Pfu pyrophosphatase is shown in FIGS. 5 and 8.

### EXAMPLE 5

Enhancement of Gene Expression by Simultaneous Co-Expression of Several tRNA Genes.

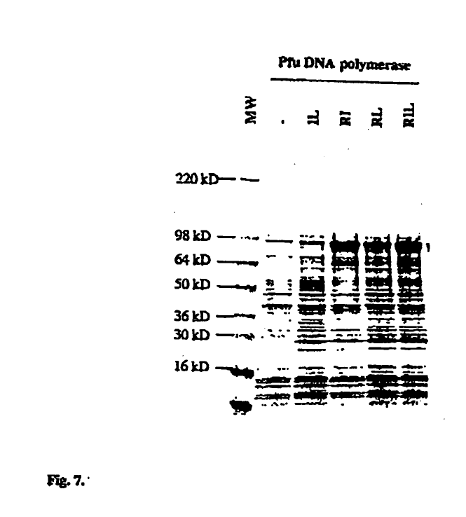

Judging by the codon usage of Pyrococcus furiosus, genes from this organism are expected to be affected by AGG/AGA (arginine) and AUA (isoleucine) codons. The sequence of Pfu DNA polymerase I contains several pairs of rare arginine and isoleucine codons. In order to test the effect of the simultaneous presence of extra copies of the argU and ileY genes on expression of Pfu-polymerase, IPTG-induced expression level of this gene was compared in strains containing extra copies of different combinations of tRNA genes. As shown in FIG. 5, expression of Pfu DNA polymerase I is enhanced by the presence of the argU gene in the RG set (argu and glyU) but to a significantly smaller degree than is achieved by a set or array containing both the argU and ileY genes (RI and RIL). Thus, simultaneous expression of argU and ileY yielded about 5-fold higher expression of Pfu DNA polymerase I than expression on either gene alone. The set containing only the ileY gene but not the argu gene (IL) failed to enhance expression of Pfu polymerase when compared to BL21goldDE3 cells. However, the argU gene in the RG set and the ileY gene in the IL set were sufficient to rescue expression of constructs only affected by AGG/AGA codons (hcTnT) or AUA codons (CBP-3xi-Cre) to the same level as was observed with the RI and RIL. The lack of efficient Pfu DNA polymerase production in host cells containing the RG set was not due to potentially negative effects of the glyU tRNA, because the same effect was observed in cells carrying the RL set (FIG. 6).

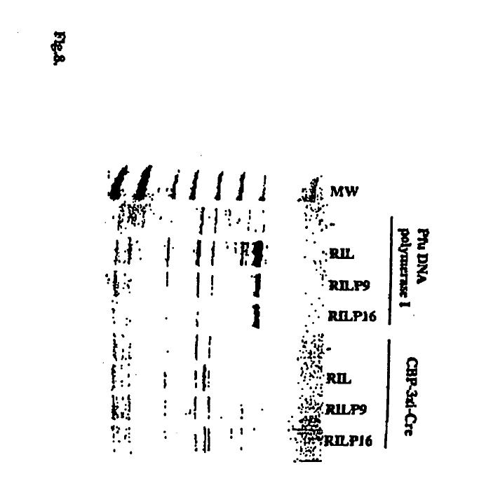

As described herein, the expression of the ileY gene is compromised in different isolates of the RILP array. Two isolates were used (RILP9, lacking detectable functional ile Y and RILP 16, displaying diminished ile Y function when compared to RIL) to demonstrate the dose-dependence of Pfu DNA polymerase expression on ileY expression. As shown in FIG. 7 the expression of Pfu DNA polymerase in the RIL and RILP strains correlates with the functional expression of ileY. Thus, efficient expression of Pfu DNA polymerase in E. coli is dependent on the simultaneous presence of extra copies of the argU as well as the ileY gene.

To test whether the improved expression of the target gene by simultaneous expression of several different tRNA genes is restricted to Pfu-polymerase 1, two additional proteins, Pfu polymerase II subunit I and subunit II were tested. Judged by their sequence, both genes should be similarly affected. In fact, expression of both genes was enhanced about 5-fold by coexpression of the argU and glyU tRNA genes (FIG. 8), similar to the observation with Pfu DNA polymerase 1. This suggests that the requirement for extra copies of two or more different tRNA genes for efficient gene expression in E. coli may be a frequent phenomenon for genes derived from organisms possessing a distinct codon usage from that of E. coli.

### Other Embodiments

Other embodiments of the invention are within the following claims.

1-44. (Cancelled)

45. A nucleic acid vector comprising sequences encoding E. coli argu, ileY and leuW tRNAs, wherein the backbone of said vector is derived from pACYC 184.

46. The vector of claim 45 wherein said sequences encoding said E. coli tRNAs are present in the order argu, ileY, leuW.

47. The vector of claim 46 wherein said sequence encoding ileY tRNA is present in the opposite orientation to said sequences encoding argu and leuW tRNAs.

48. The vector of claim 45 that comprises a tet promoter.

49. The vector of claim 48 wherein said tet promoter is operably linked to a sequence encoding a said tRNA.

50. The nucleic acid vector of claim 45 wherein said sequence encoding E. coli argu tRNA comprises the sequence between base pairs 8041 and 8260 of GenBank Accession No. AE000159.

51. The nucleic acid vector of claim 45 wherein said sequence encoding E. coli ileY tRNA comprises the sequence between base pairs 7741 and 7950 of GenBank Accession No. AE000350.

52. The nucleic acid vector of claim 45 wherein said sequence encoding E. coli leuW tRNA comprises the sequence between base pairs 241 and 378 of GenBank Accession No. J01713.

53. The nucleic acid vector of claim 45 which further comprises a chloramphenicol resistance gene.

54. A nucleic acid comprising a promoter operably linked to sequences encoding E. coli argu, ileY and leuW tRNAs.

55. The nucleic acid of claim 54 wherein said sequences encoding E. coli tRNAs are present in the order argu, ileY, leuW.

56. The nucleic acid of claim 55 wherein said sequence encoding ileY tRNA is present in the opposite orientation to said sequences encoding argu and leuW tRNAs.

57. The nucleic acid of claim 54 wherein said promoter is the tet promoter.

58. The nucleic acid of claim 54 wherein said promoter is endogenous to a said sequence encoding an E. coli tRNA.

59. The nucleic acid of claim 54 wherein said sequence encoding E. coli argU tRNA comprises the sequence between base pairs 8041 and 8260 of GenBank Accession No. AE000159.

60. The nucleic acid of claim 54 wherein said sequence encoding E. coli ileY tRNA comprises the sequence between base pairs 7741 and 7950 of GenBank Accession No. AE000350.

61. The nucleic acid vector of claim 54 wherein said sequence encoding E. coli leuW tRNA comprises the sequence between base pairs 241 and 378 of GenBank Accession No. J01713.
